(19) United States
(12) Patent Application Publication
(10) Pub. No.: US 2007/0208343 A1
(43) Pub. Date: Sep. 6, 2007

(54) Implant Used in Stabilising Operations on the Thoracic and Lumbar Vertebral Column

(75) Inventors: Fritz Magerl, St. Gallen (CH); Roger Stadler, Zurich (CH)

Correspondence Address: VOLPE AND KOENIG, P.C., UNITED PLAZA, SUITE 1600, 30 SOUTH 17TH STREET, PHILADELPHIA, PA, 19103, US

(73) Assignee: Sepitec Foundation, Vaduz (CH)

(21) Appl. No.: 10/592,406

(22) PCT Filed: Mar. 10, 2004
(86) PCT No.: PCT/EP04/02466

§ 371 Date: Sep. 11, 2006

(51) Int. Cl. A61B 17/58

(52) U.S. Cl. 606/61

(57) ABSTRACT

An implant used in stabilising operations on the thoracic and lumbar vertebral column for restoring the load-bearing capacity of the vertebral column is provided. An insertion region of the implant (I) comprises a wedge-shaped distraction part (C), which permits the vertebral bodies to be pushed apart in order to increase the intervertebral distance. In addition, guide elements in the form of grooves (7) and/or strips are provided on said wedge-shaped distraction part (C) and/or in the implant part (D).

### BACKGROUND

The invention relates to an implant used in stabilizing operations on the thoracic and lumbar vertebral column for restoring the load-bearing capacity of the vertebral column.

In a known intervertebral implant DE 200 04 693 U1, the vertical distance of the vertebrae can be enlarged by rotating the implant in the intervertebral-disc space. The vertical distance is increased by the difference between the implant width and the implant height if the elongated right parallelepiped-shaped vertebral implant has been inserted into the intervertebral-disc space with its side walls parallel to the end plates of the vertebrae and is then rotated 90 degrees about its longitudinal axis with the help of the insertion instrument attached to the implant. A prerequisite for the distracting effect of the mentioned mechanism is that the implant is taller than it is wide. Other possibilities for enlarging the vertical distance are not disclosed by publication. The relevant vertebra implant has vertical end and side walls and a central recess continuous in the height direction, wherein the end walls are significantly thicker than the side walls for enlarging the contact surface with the marginal rings of the vertebrae. In the side view, the upper and lower edges of the side walls have a continuously convex curve up to the end of the implant. Such a shaping of the contact surfaces to the vertebrae is also to be found in other intervertebral implants and is used only to improve the contact of the side walls and the transplant material filled into the implant hollow space with concave vertebral end plates. The edges of the implant are rounded in order to prevent the risk of injuring important structures surrounding the implant.

In another known intervertebral implant (WO 01/54 620 A1) that can be produced from various material with differently structured contact surfaces to the vertebrae can also be inclined differently relative to these surfaces, whereby the implant overall can obtain a more or less pronounced wedge shape. The wedge shape of the implant is obviously not used for enlarging the intervertebral distance, especially since it is mentioned several times that the rear end of the implant should be higher than the front. If one assumes that the end of the implant, with which it is brought into the intervertebral-disc space, is typically designated as the front end, the wedge shape appears to have a function other than a distracting function. Obviously, it is used for restoring a forwards convex curvature of the lumbar vertebral column. If the implant is to be distracted to a significant degree, it must have an additional wedge with greater incline at its front end.

Furthermore, an implant made from preserved human or animal bone or from another biocompatible material and with a wedge shape in the side view and a ring, C, or rectangular shape in the top view, is known (WO 00/74 608 A1). In this case, the selectable wedge shape is also used only for restoring the natural curvature of the vertebral column.

### SUMMARY

The invention has set for itself the challenge of creating compression-proof implants, which can be inserted into the intervertebral-disc spaces of the thoracic and lumbar vertebral column from the rear, from the rear on the side, or from the side, which can be implanted optimally, which also can provide a distraction effect itself, and which also enables the load capacity of the vertebral column to be restored.

This is achieved according to the invention in that the implant, at its insertion region, has a wedge-shaped bevel, which enables the vertebra to be pressed apart for enlarging the intervertebral distance.

A special advantage of the implant according to the invention is that the vertical distance of the vertebrae can be enlarged by the wedge-shaped insertion part of the implant.

The invention considerably simplifies the surgical technique of dorsal, dorsolateral, or lateral implantation of intervertebral implants and improves the safety and also the success rate of these techniques. Apart from the typical function of an intervertebral implant, the use of the intervertebral implant according to the invention offers the following advantages:

A tight lodging of the implant within the intervertebral-disc space is achieved in order to secure the stability necessary for the osseous consolidation of spondylodesis. The enlargement of the vertical distance of the vertebrae to be stabilized (distraction) is enabled, in order to obtain the necessary tension on the soft tissues for lodging the implant and also to achieve expansion of the spinal canal and the intervertebral foramina (decompression), which accompanies distraction. It is further proposed that the implant is formed from a wedge-shaped insertion part (distraction part C) and a pressure-transmitting implant part (D). At least the end phase of the distraction, which is decisive for the lodging of the implant, can be produced with the implant itself. For biomedical reasons, the longitudinal axis of the implant inserted completely into the intervertebral-disc space can lie in the frontal plane.

Here, it is advantageous when the angle of the wedge surfaces equals at least approximately 30°, preferably 25° to 30°. Therefore, this produces good possibilities for inserting the implant without the risk of injury.

An especially advantageous configuration is that guide elements, in the form of grooves and/or ridges are provided in the wedge-shaped end part and/or in the implant part. In this way, in a special construction, grooves are machined into the top and bottom surfaces of the pressure-transmitting implant part bordering the recess on the wedge side. These grooves have sharp edges and run parallel or concentric to the sharp front edges, optionally, the grooves or the ridges set on these edges are also straight. In a special configuration it is also provided that the grooves have the shape of an inverted lean-to roof-like, dihedral groove, whose rear surface is perpendicular to the top surface of the implant and forms a sharp edge with this surface and whose front surface rises at an angle relative to the top surface of the implant.

Thus, additional features of the invention are special configurations, which automatically guide the implant from the sagittal or inclined insertion direction into the intended frontal end position when the implant is inserted into the intervertebral-disc space. Therefore, the implant is made so that it can rotate in the intervertebral-disc space from the sagittal or inclined implantation direction into the transverse end position. Since the desired secure lodging of the implant restricts its ability to rotate or makes this motion impossible, the implant according to the invention is equipped with the guidance device, which has the effect that the implant rotates by itself into the transverse end position during the insertion into the intervertebral-disc space. Nevertheless, even if the transverse end position is not achieved automatically, a completion of the rotation is made possible. By the special configuration, it is also possible to keep the surface pressure, in the regions of the contact surfaces between the vertebrae and the implant, as small as possible by shaping the surfaces of the implant as large as possible. The implant can be implanted from behind (PLIF, TLIF), from the side, or with the help of the new technique (EPLIF) to be explained in more detail below diagonally from behind using a biportal or uniportal method.

### BRIEF DESCRIPTION OF THE DRAWINGS

Additional features are contained in the subordinate claims and also in the following description. In the following description, embodiments of the invention are explained in more detail with reference to the drawings. Shown are:

FIG. 1, an embodiment for an intervertebral distraction implant in a view from above;

FIG. 2, a section along line II-II in FIG. 1;

FIG. 3, a section along line III-III in FIG. 1;

FIG. 4, a section along line IV-IV in FIG. 1;

FIG. 5, a section along line V-V in FIG. 1;

FIG. 6, another embodiment for an intervertebral distraction implant in a view from above;

FIG. 7, a section along line VII-VII in FIG. 6;

FIG. 8, a section along line VIII-VIII in FIG. 6;

FIG. 9, a section along line IX-IX in FIG. 6;

FIG. 10, a section along line X-X in FIG. 6;

FIG. 11, another embodiment for an intervertebral distraction implant in a view from above;

FIG. 12, a view from behind;

FIG. 13, a section along line XIII-XIII through a guide groove;

FIG. 14, a view of the configuration of FIG. 11 from the side (implant rear portion);

FIG. 15, a perspective view of the configuration from FIGS. 11 to 14;

FIG. 16, a further embodiment for an intervertebral distraction implant in a view from above;

FIG. 17, a view of the implant of FIG. 16 from the side (implant front end);

FIG. 18, a view from the front;

FIG. 19, a section along line XIX-XIX in FIG. 16;

FIG. 20, a section along line XX-XX in FIG. 16;

FIG. 21, a perspective view of the configuration from FIGS. 16 to 20;

FIGS. 22 and 23, representations of the vertical extension of an intervertebral-disc space with a temporarily positioned implant (FIG. 22) and with the implant then inserted into the intervertebral-disc space (FIG. 23);

FIGS. 24 to 27, the implantation of an intervertebral distraction implant, wherein the figures show the phases I to IV of the procedure of an implantation;

FIGS. 28 and 29, a distraction instrument viewed from above and from the front.

First a few basic comments will be given that are important with regard to the present invention:

Spondylodesis is a connection of vertebrae, wherein this connection comprises mostly bone and permanently blocks the movement of the connected vertebrae. To create spondylodesis, materials stimulating osteogenesis, such as bone shavings or bone replacement materials are either inserted between the vertebrae (interbody spondylodesis) or placed above the rear vertebral elements (dorsal spondylodesis). A prerequisite for the conversion of these materials into rigid bone is that there are no movements in the relevant section of the vertebral column disrupting the conversion process during the osteogenesis. To guarantee this, stabilizing implants are often used.

Pains that are not affected by conservative treatment measures, spinal cord or nerve root compression, as well as dislocations are indications for interbody spondylodesis. In principle, pain can come from any pathologically changed structure of the vertebral column. Instability is the term that is used when vertebrae can no longer move properly relative to each other due to such changes. Prolapsed intervertebral discs and the narrowing of the vertebral canal or the intervertebral foramina are responsible for the development of spinal cord or nerve root compression. In the scope of spondylodesis, the causes of compression syndromes are also rectified (decompression).

Because the intervertebral discs are always cleared out in interbody spondylodesis and this severely affects the stability of the vertebral column negatively, their ability to bear weight must always be restored. This can happen, e.g., by the use of compression-proof bone blocks, which are taken from the patient (autogenous bone blocks) and inserted between the vertebrae. However, since the ability of such shavings to bear weight is often unreliable and their availability is limited and also because the morbidity caused by shaving removal can be considerable, increasingly bone replacement materials created from materials foreign to the body are being used together with intervertebral implants (“cages”) instead of autogenous bone blocks.

The spondylodesis technique has to satisfy, in particular, the following requirements: reduced distances between the vertebrae caused by tapering intervertebral discs are to be normalized and vertebral displacements and also curvature of the vertebral column are to be rectified. Because the enlargement of the vertical vertebral distance by “distraction” already has a strong decompressing effect, distraction is usually an essential component of the surgery. With each distraction acting in the regions of the vertebrae, an existing kyphotic curvature of the relevant section of the vertebral column is reduced or prevented, such that bending is realized in the scope of the spondylodesis. This effect can be supported by a slightly wedge-shaped form of the intervertebral implant in the sagittal direction. In a conventional way, the distraction is realized with the help of special instruments, with pedicle systems, or by rotating special intervertebral implants by 90 degrees in the intervertebral-disc space.

Implantation techniques for intervertebral implants are to be used in the following way:

Intervertebral implants can be inserted into all regions of the vertebral column from the front (ALIF—anterior lumbar interbody fusion); also from behind on the lumbar vertebral column (PLIF—posterior lumbar interbody fusion), (TLIF—transforaminal lumbar interbody fusion), from the side, or from the rear on the side. For each intervertebral-disc space, one or two implants are used. Biportal implantation is the term used when two implants are inserted from behind through two separate openings of the intervertebral-disc ring. For a uniportal implantation (cf. Fig.) the intervertebral disc is opened at only one position.

In the following, when intervertebral implants are discussed, these refer to compression-proof, non-elastic implants.

A compression-proof intervertebral implant functions as a place holder, which transfers loads from the upper to the lower vertebra, ensures the vertical distance of the vertebrae relative to each other, and guarantees that a solid bone bridge can form between the vertebrae. Bone or bone replacement material filling up the implants and/or placed around the implants or just the blood collecting in the intervertebral-disc space form the matrix for osteogenesis. For the ossification process, the stability of the spondylodesis plays a decisive role, because movements occurring between the intervertebral implant and the vertebrae can prevent the osseous consolidation of the interbody spondylodesis. Furthermore, the intervertebral implant can sink or penetrate into the vertebrae. To prevent this result, the spondylodesis is usually stabilized by implants anchored ventrally or dorsally to the vertebrae.

In addition to therapeutic significance, distraction also has a considerable mechanical significance: the expansion of the intervertebral discs and the intervertebral-disc ring accompanying the distraction generates a counter force, which lodges the intervertebral implant between the vertebrae. This lodging force also prevents harmful movements between the vertebrae and the implant and also reduces the risk of sometimes serious secondary implant dislocations.

For the implantation of intervertebral implants, the distraction is typically achieved through the aid of special devices (e.g., distraction instruments, pedicle system) or by inserting an intervertebral implant, whose vertical side wall height is greater than the width of the horizontal top and bottom surfaces, into the intervertebral-disc space such that the walls first contact the end plates of the vertebrae. If the implant is then rotated by 90 degrees about its longitudinal axis, the intervertebral space expands by the difference between the height and the width. Because the width must be smaller than the height, this technique has the disadvantage that the sizes of the surfaces transferring the axial pressure of the vertebral column from the vertebrae to the intervertebral implant must be relatively small. However, for small contact surfaces, there is the risk that the intervertebral implant will penetrate into the vertebrae due to the high surface pressure.

First an overview of the various construction features will be given. Then, at the end of the description, the drawing figures will be discussed in more detail.

Overall, the implant body I is preferably kidney-shaped or bean-shaped. Its longitudinal diameter is greater than its transverse diameter. The front implant wall 10a has a convex curve in the longitudinal direction of the implant. The rear implant wall 10b can be straight or have a concave curve in this direction. Viewed from above, the front and rear wall transition with a curved form into the other at the distraction wedge C and at the opposite implant end, so that the relevant ends of the implant are rounded in a top view. In the direction from top to bottom, the front implant wall 10a and the rear implant wall 10b are vertical to a horizontal plane cutting the implant in half. The implant I can have a central recess 6 from its top surface 2a to the bottom surface 2b or it can be solid (not shown). A special feature of the distraction implants according to the invention, which can be configured differently in detail, is the provision of various guide elements, such as grooves 7, sharp-edged ridges 9 set thereon, specially shaped implant edges 3a, 3b, 4, and also sharp-edged ridges 15 at the front edges of the implant.

In terms of function, the implant consists of two parts: the distraction wedge C and the compression-proof implant part D transmitting the pressure from one vertebra to another.

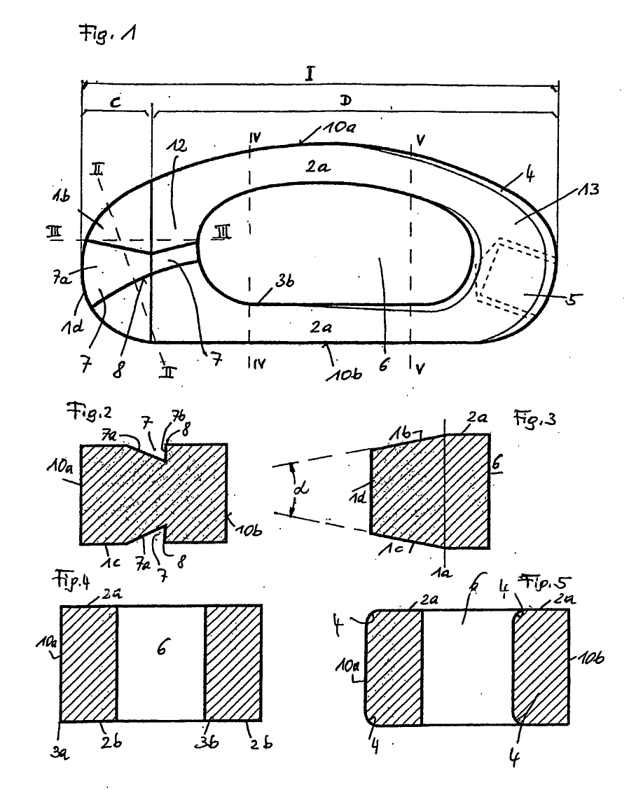

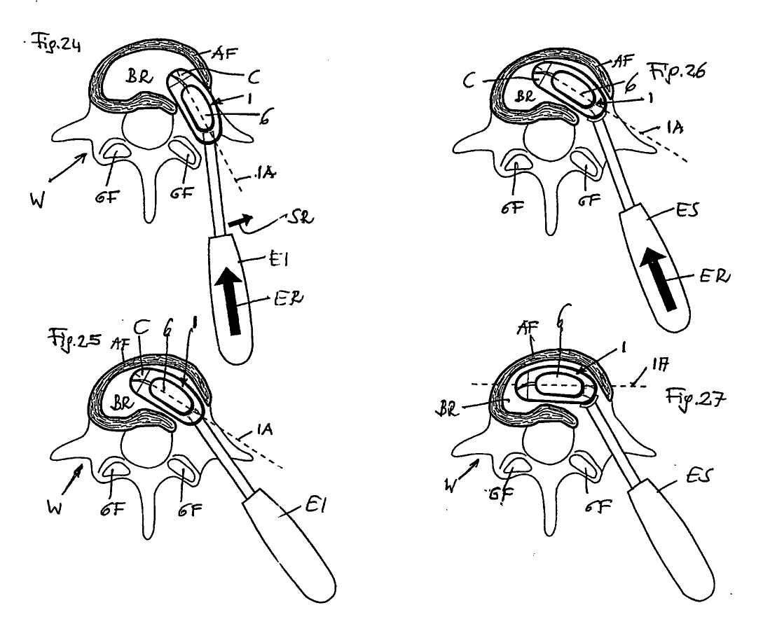

With the distraction wedge C, the intervertebral-disc space is expanded vertically by inserting the implant. This part of the implant has a wedge-shaped outline. The base 1a of the wedge connects the distraction wedge C to the implant part D and has the same height as the implant part D. The surface of the base 1a can vertical (FIGS. 1, 6, 11) or at an angle (FIG. 16) to the longitudinal axis of the implant. The difference in height between the wedge base 1a and the front wedge end 1d preferably equals ca. 3 mm. The implant then increases the height of the intervertebral-disc space by 3 mm. Aside from the special guide elements 7, 7a-b, 8, 9 the flat wedge surfaces 1b, 1c are inclined relative to the wedge end 1d, which is vertical in its elevation plan and rounded in its base plan, such that the wedge end is lower than the wedge base. The angle α defined by the wedge surfaces 1b, 1c (FIGS. 3, 8, 12, 20) preferably equals between 25° and 30°. At the wedge base 1a, the wedge surfaces 1b, 1c of the distraction wedge C transition with a slight rounding 14 (shown only in FIGS. 16 and 21) and continuously into the top or bottom surface 2a, 2b of the pressure-receiving implant part D. Both wedge surfaces 1b, 1c can each feature guide grooves 7, which run in parallel or concentric to the sharp front edges 3, 3b of the wedge end 12 of the implant part D and which continue into the guide grooves 7 formed at the top and bottom surface of the wedge-side part of the implant part D. Furthermore, straight grooves are also contemplated, which run through the surfaces of the implant, such that their direction corresponds approximately to a tangent positioned in a top view at the transition of the implant part D into the distraction wedge C to the curvature of the front wall 10a. In the side view, the tangent runs parallel to the longitudinal axis of the implant. The grooves preferably lie in the center of the surfaces 1b, 1c (FIGS. 1, 6, 11) but can also be formed farther towards the back (FIG. 16).

The parts of the guide grooves 7 or ridges 9 formed on the distraction wedge C form guide elements and can have various shapes:

In cross section the groove 7 has the shape of an inverted lean-to roof with two surfaces. The vertical rear wall 7b of the groove forms a sharp edge 8 with the rear portions of the wedge surfaces 1b, 1c. The front groove surface 7a forms an acute angle with the groove rear wall 7b in the groove depth direction and rises from the base at an angle towards the front portions of the wedge surfaces 1b, 1c. The groove can have the same width and the same depth overall. At the wedge base 1a, it transitions into the groove of the implant part D. This groove then has, in its longitudinal profile at the transition from the distraction wedge C into the implant part D, a bend corresponding to the angle of the relevant wedge surface 1b or 1c relative to the top or bottom surface of the implant part D.

Furthermore, the groove 7b formed on the distraction wedge C can be shaped such that the groove becomes gradually wider towards the wedge end 1d away from the wedge base 1a (FIG. 1). In this case, the front groove surface 7b has a smaller incline relative to the appropriate top surface 1b or 1c of the wedge end C than the corresponding groove surfaces of the implant part D. The height of the vertical groove rear wall 7b and thus also the groove depth can remain unchanged.

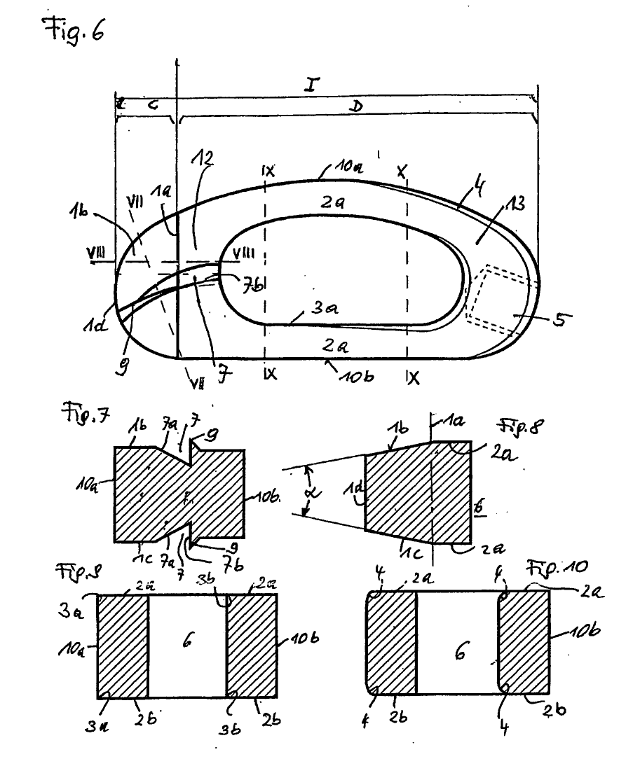

As an additional guide element, a ridge 9 (FIGS. 6, 7) with a lean-to roof-shaped cross section can be formed on the distraction wedge C such that its vertical front wall continues the vertical rear wall 7b of the guide groove 7. The rear wall of the ridge falls at an angle relative to the relevant top surface 1b or 1c of the distraction wedge C. In this configuration, the ridge has a sharp top edge 9. The ridge can begin at the wedge end 1d and end at the wedge base, in that its height decreases continuously from the wedge end 1d to the wedge base 1a, such that at the wedge base 1a, the implant part D no longer juts out. However, in the same lean-to roof-shaped configuration, which it has in distraction part C, the ridge can also continue into the implant part D. In this case, the ridge then projects over the top surfaces 2a, 2b of the implant part D.

The edges formed by the wedge surfaces 1b, 1c and the vertical walls 2a, 2b, 1d connecting them vertically can be rounded in the front half of the distraction wedge C or can be sharp like the front edges 3a of the implant part D. In the rear part of the distraction wedge C, the relevant edges are rounded. In top view, the implant part D has a bean or kidney shape and can have a central recess 6 passing through the implant in the vertical direction for receiving bone or bone replacement material or can be compact and can feature a device for attaching an implantation instrument (5).

The top and bottom surfaces 2a, 2b of the implant part D run parallel to each other in the longitudinal direction. In this direction, they can be flat or convex and in the direction from the back to the front they can be flat or have a slight convex curve, as well as parallel to each other or inclined relative to each other. The front implant wall 10a has a convex curve in the longitudinal direction of the implant. The implant rear wall 10b can be straight or have a concave curve in this direction. In the direction from top to bottom, the vertical implant walls 10 can be flat, convex, or concave, as well as closed or can feature openings that are continuous from the outside into the central recess. The front edges of the implant part D become sharp 3a, 3d towards the wedge end 12 and increasingly round towards the opposite end 13. Sharp-edged guide ridges 15 somewhat projecting from the edges of the front wall 3a are formed on the wedge end 12 of the implant part D (FIGS. 16-21). A screw or plug device 5 for attaching an insertion instrument can be formed on the wedge end opposite the implant part 13. At the wedge end 12 of the implant part D, in the top and bottom surface 2a, 2b of the implant part D, a groove 7 with a lean-to roof-shaped cross section (FIG. 11) can be formed, which continues the corresponding groove of the distraction wedge C into the implant part D. Aside from the possible widening (FIG. 1) in the distraction wedge C towards the wedge end, the grooves can be formed like the grooves in the distraction wedge C. The sharp edges 8 of the grooves run parallel to the sharp front edges 3a. However, their curvature can also correspond to an arc concentric to the sharp front edges 3a. Furthermore, straight grooves (not shown) are conceivable, which run at an incline through the top surfaces 2a, 2b of the implant. Their direction should correspond to that of a tangent positioned in a top view at the transition of the implant part D into the distraction part C to the curvature of the front wall 10a. In the side view, the tangent runs parallel to the longitudinal axis of the implant.

Another possible shape of the guide elements consists in not only the groove 7 continuing from the distraction wedge C into the implant part D, but also a sharp-edged ridge 9 on the groove. This ridge 9 then projects over the surfaces 2a, 2b of the implant part D. The shape of the distraction wedge C is essential for the success of the distraction caused by the intervertebral implant itself.

According to the invention, the distraction generated by the intervertebral implant itself is achieved in that the distraction wedge C, that is, the part of the implant, with which it is inserted into the intervertebral-disc space, is wedge-shaped. The distraction wedge C of the intervertebral implant expands the intervertebral-disc space by the difference between the wedge base 1a and the wedge end 1d, e.g., by 3 mm. To ensure success of the distraction, it is essential that the surfaces 1a, 1b forming the wedge are flat, aside from the guide elements formed there, and that the angle α defined by the wedge surfaces 1a, 1b (FIGS. 3, 8, 12, 20) is not too large. Preferably, it should be no larger than ca. 30 degrees. At a larger angle there would be the risk that the edges would penetrate the vertebrae when the distraction wedge C is inserted. A convex bend of one or two wedge surfaces or a completely spherical shaping of the insertion part, as is the case in a few known intervertebral implants, would also not be favorable. If an implant with a convex or a spherical insertion part is pressed into an intervertebral-disc space and is a few millimeters smaller than the implant height, the insertion part first meets the edges of the vertebrae at a great slope. These edges can break, when, as is the case analogously for the intervertebral implant according to the invention, the implant must be pressed or hammered into the intervertebral-disc space against sometimes considerable resistance (cf. FIG. 22). Therefore, such a shaped intervertebral implant cannot be used without risks in the sense of the invention discussed here as an actual distraction implant.

For disorders of the intervertebral discs, the height and also the vertical expandability (distractibility) of the intervertebral-disc space can vary greatly. The height of the intervertebral-disc space that can be achieved by distraction is the decisive factor for the height of the intervertebral implant to be implanted. The intervertebral implant must be adjustable to this height. To enable this, it is provided to make the intervertebral implant available in various heights. The height difference between the individual implants and the shape of the distraction wedge can increase linearly (e.g., by 3 mm each time) or non-linearly from the minimum to the maximum height.

The distractibility of the intervertebral-disc space can already be seen in the removal of the intervertebral disc tissue, which is always necessary for the implantation of the intervertebral implant, for the movement of the vertebrae bordering the intervertebral-disc space. The two vertebrae can always be connected tautly to each other, such that a very strong resistance is to be overcome during the distraction attempt. However, it can also be that a considerable vertical expandability of the intervertebral-disc space can be discernible in the distraction attempt.

For taut connection of the vertebrae, the distractibility of the intervertebral-disc space is low. In this case, distraction is done only with the implant. The implant is selected, whose wedge end 1d can be inserted straight into the intervertebral-disc space. The distraction resulting from hammering the implant into the intervertebral-disc space is then sufficient for its stable lodging.

To avoid having to remove an already inserted implant and replacing it by a higher implant, the intervertebral-disc space is distracted for clear vertical expandability with the help of special distractors (FIGS. 28 and 29) incrementally until it is discerned from the resulting resistance that the limit of distractibility has been nearly reached. The implant is then inserted, whose wedge end (1d) can be inserted straight into the intervertebral-disc space. The last distraction step securing a stable lodging of the implant between the vertebrae is realized with the intervertebral implant itself.

The goal of stable lodging of the intervertebral implant can only be reached when the discs of the vertebral column responsible for the lodging force and external parts of the intervertebral disc are tensioned but not torn. This requires a dosed and controllable distraction. For this purpose, a set of special distractors is provided (FIGS. 28 and 29), with which the intervertebral-disc space can be expanded (distracted) step by step in the vertical direction. In terms of function, the bodies of the distractors 16 can comprise two parts, wherein the distracting part is shaped similar to the intervertebral implant according to the invention and also has, in particular, a distraction wedge 17. The heights 19 of the distractors are matched to the intervertebral implants, such that each distractor enables the insertion of the distraction wedge C of the matching intervertebral implant into the intervertebral-disc space. According to the invention, this is achieved in that the wedge base 1a and body 16 of the distractor are each higher by approximately, e.g., 1 mm, than the wedge end 1d of the matching intervertebral implant. Furthermore, the distractor has a handle 18 connected to the distracting part for inserting and hammering the distractor into the intervertebral-disc space. Here, each distractor can have a handle connected rigidly or by a plug or screw connection to its body.

In the direction of its longitudinal axis, the intervertebral implant can be inserted diagonally from the rear (“dorsolateral”) (see FIGS. 24 to 27), sagittally from the rear (“dorsal,” PLIF or TLIF), or from the side into the intervertebral-disc space. In the end position in the intervertebral-disc space, the longitudinal axis of the implant must run in the transverse direction, i.e., it must lie in the frontal plane. Except for a purely lateral implantation direction, the implant must be rotated from the dorsolateral or dorsal position into the transverse end position during its insertion into the intervertebral-disc space, so that its longitudinal axis finally lies in the frontal plane. However, for the desirable stable jamming of the implant, its rotation in the intervertebral-disc space can present considerable difficulties or can be impossible, if special precautions are not taken that make the rotation possible.

The invention should simplify the insertion of the intervertebral implant into the transverse end position. According to the invention, this is achieved by providing the intervertebral implant with special guide elements, which rotate the implant into the end position when it is inserted.

The guide elements comprise, on the distraction wedge C, the grooves 7 and optional ridges 9 and, on the implant part D, the sharp edges 3a, 3b, the grooves 7, the ridges 9 possibly positioned on the grooves, and the ridges 15 possibly positioned on the sharp front edges 3a. The front edges 3a, 3b of the implant are bent according to the sector of an arc, in that the front wall 2a of the implant represents the sector of a round cylinder, whose axis lies close to the center of the spinal canal. The curvature of the grooves 7 and the ridges 9 possibly positioned on the grooves corresponds to an arc concentric to the front edges.

The counter pressure arising during the distraction, presses the end plates of the vertebrae against the intervertebral implant. Therefore, when the implant is inserted, the guide elements cut into the end plates of the vertebrae. The curvature of the edges has the effect that the implant rotates by itself along an arc into the end position during the insertion into the intervertebral-disc space.

By the use of, for example, a bar-shaped insertion instrument EI attached to its rear portion 13, the implant can also be initially guided. The instrument is retracted as soon as it has reached the border of the insertion opening lying in the intervertebral-disc ring and therefore can not be pivoted any further. The implant is further hammered by a ram ES positioned on its rear portion 13 and, if necessary, simultaneously rotated with the ram into the final position.

This final rotation into the transverse position is possible because the guide elements are attached only to the insertion part of the intervertebral implant and the front edges of the implant are rounded towards the other rear end of the implant. The implant (FIGS. 26, 27) is no longer urged in the direction of its longitudinal axis, but instead is hammered at an angle to this axis, the part of the implant carrying the guide element does always enter along the arc in the direction given by the guide element, but the rear portion of the implant 13 can be forced out of the path towards the front thanks to the rounded front edges 4 and the lack of the guide element. Here it pivots forwards about a vertical axis in the region of the insertion part.

If the intervertebral implant is inserted from the side into the intervertebral-disc space, it does not have to be rotated. In this case, its longitudinal diameter already lies in the frontal plane when it is inserted. Intervertebral distraction implants (not shown) designed for a side application do not necessarily have to be equipped with guide elements.

To reduce the pressure acting on the contact surfaces of the implant with the vertebrae, the invention further intends to insert the largest possible implant into the intervertebral-disc space. Because access to intervertebral discs can be very narrow for the use of typical access paths through the spinal cord canal from the dorsal side (PLIF, TLIF), an access (FIGS. 24 to 27) is recommended through which implants with larger transverse diameter can be inserted. The already known new access leads to the outer side of the intervertebral foramina (FIGS. 24 to 27) under the mass of the extensor muscles in the back. The intervertebral-disc ring is opened from there by the roots of the arc (extraforaminal submuscular access). The opening can be expanded such that implants with a larger transverse diameter can be inserted than those for access through the spinal cord canal. The implant with the largest possible width should always be used.

Intervertebral distraction implants should be able to be inserted in all areas of the thoracic and lumbar vertebral column into the intervertebral-disc space dorsally, dorsolaterally, or laterally. Therefore, it is provided to match the shape of the implant parts both to the appropriate anatomical conditions and also to the provided implantation technique by adjusting, among other things, the curvature of the guide element, e.g., for purely lateral or purely dorsal implantation. Furthermore, different size relationships both in the individual regions of the vertebral column and also the accesses into the intervertebral discs require that the intervertebral implants must be made available also in different sizes adapted to the application site and to the application technique.

Intervertebral distraction implants can be composed of metal, polymer, or composite material. Intervertebral implants made from polymer or composite material are not visible radiologically. To make them visible, elements making radiological shadows are installed in the implants. It is provided to equip the implant according to the invention with radiological shadow-forming elements, e.g., with very thin tantalum wires. The surfaces of the implants can also be structured and/or coated, wherein structures projecting from the surfaces are formed, such that they form rows running preferably in parallel or concentric to the front edges of the implant, in order to be able to also act as a guide element.

### DETAILED DESCRIPTIONS OF THE PREFERRED EMBODIMENTS

The following discusses the drawing figures in detail:

FIGS. 1 to 5 show an embodiment for an intervertebral distraction implant with guide grooves becoming wider towards the front. Here, FIG. 1 is a view of the implant from above. The implant I comprises a pressure-receiving part D and the distraction wedge C. In the elevation plan, it has a convex front wall 10a, a straight rear wall 10b, and is rounded on the distraction wedge C and the opposite end 13. The implant walls enclose a central recess 6. The front edges of the implant part D become increasingly sharp 3a, 3b towards the distraction wedge C and are increasingly rounded 4 towards the opposite end 13. At this end of the implant part D there is a bore 5 for attaching an insertion instrument. In the top 1b and bottom surface 1c of the distraction wedge C, and also in the bordering surfaces 2a, 2b of the implant part D, grooves 7 are formed running in parallel or concentric to the front edges 3a. In this embodiment, the grooves formed in the center parts of the wedge surfaces 1b, 1c and the bordering surfaces 2a, 2b of the implant part D become wider towards the wedge end 1d.

From FIG. 2 the groove 7, in particular, can be seen. It has the shape of an inverted lean-to roof in cross section and accordingly, a vertical rear wall 7b and a front surface 7a rising diagonally towards the surfaces 1b, 1c of the distraction wedge C. The rear wall 7b of the groove forms a sharp edge 8 with the surface of the distraction wedge C. The front wall 10a and rear wall 10b of the distraction wedge C extend vertically and at a right angle to the surfaces 1b, 1c.

FIG. 3 shows a longitudinal section through the distraction wedge. The top wedge surface 1b and the bottom wedge surface 1c define the angle α, which should not be larger than 30 degrees. The surfaces 1b, 1c, forming the wedge, transition at the wedge base 1a with a bend into the surfaces 2a, 2b of the part 12 of the implant part D bordering the distraction wedge. The appropriate transition can be rounded by a small radius 14 (not shown here, cf. FIGS. 16, 20, 21). The surface opposite the wedge end 1d borders the central recess 6.

In a section, FIG. 4 primarily shows the front edges 3a, 3b becoming sharp at the wedge end 12 of the implant part D. They are formed by the surfaces 2a, 2b and the front surfaces of the front wall 2a and rear wall 2b of the implant part D. The front 2a and rear wall 2b enclose the central recess 6 that is continuous from top to bottom.

The section from FIG. 5 shows the front edges 4 becoming increasingly round towards the implant end 13 opposite the distraction wedge C. Incidentally, the section is to be described the same as the section shown in FIG. 4.

The embodiment shown in FIGS. 6 to 10 shows an intervertebral distraction implant with guide ridges positioned on the guide grooves. In the view of FIG. 6, it can be determined that the implant I comprises a pressure-receiving part D and distraction wedge C. In the elevation plan, it has a convex front wall 10a, a straight rear wall 10b, and is rounded on the distraction wedge C and opposite end 13. The implant walls enclose a central recess 6. The front edges of the implant part D become increasingly sharp 3a, 3b towards the distraction wedge C and are increasingly rounded 4 towards the opposite end 13. At this end 13 of the implant part D, there is a bore 5 for attaching an insertion instrument. In the top surface 1b and bottom surface 1c of the distraction wedge C, as well as in the bordering surfaces 2a, 2b of the implant part D, grooves 7 running in parallel or concentric to the front edges 3a are formed. In this embodiment, the grooves become more shallow towards wedge 1d. Here, as another guide element, ridges 9 are formed, which have a lean-to roof-shaped cross section and which project from the wedge surfaces 1b, 1c no higher than up to the level of the surfaces 2a, 2b of the implant part D, begin at the wedge end 1d, and become smaller towards the wedge base 1a, such that they no longer project over the surfaces 2a, 2b of the implant part D adjacent to the distraction wedge C. The ridges continue in the direction of the grooves up to the wedge end 1d. In this case, the grooves 7 and the ridges 9 also lie in the middle parts of the wedge surfaces 1b, 1c and the adjacent surfaces 2a, 2b of the implant part D.

FIG. 7 illustrates the shape of the grooves 7 and the lean-to roof-shaped, sharp-edged ridges 9 positioned on these grooves. Each groove has, in cross section, the shape of an inverted lean-to roof and accordingly a vertical rear wall 7b and a front surface 7a rising at an angle relative to the surfaces 1b, 1c of the distraction wedge C. Here, however, the rear wall 7b of the groove continues into the front wall of the ridge, such that both walls form a flat surface standing vertical to the wedge surfaces 1b, 1c. This surface forms a sharp edge pointing forwards with the surface falling at an angle relative to the appropriate surface 1b or 1c of the distraction wedge C. The front wall 10a and rear wall 10b of the distraction wedge C extend vertically and at a right angle to the surfaces 1b, 1c.

FIG. 8 shows a longitudinal section through the distraction wedge. The top wedge surface 1b and the bottom wedge surface 1c enclose the angle α, which should not be greater than 30 degrees. The surfaces 1b, 1c forming the wedge transition at the wedge base 1a with a bend into the surfaces 2a, 2b of the part 12 of the implant part D adjacent to the distraction wedge. The relevant transition can be rounded by a small radius 14 (not shown here, cf. FIGS. 16, 20, 21). The surface opposite the wedge end 1d defines the central recess 6.

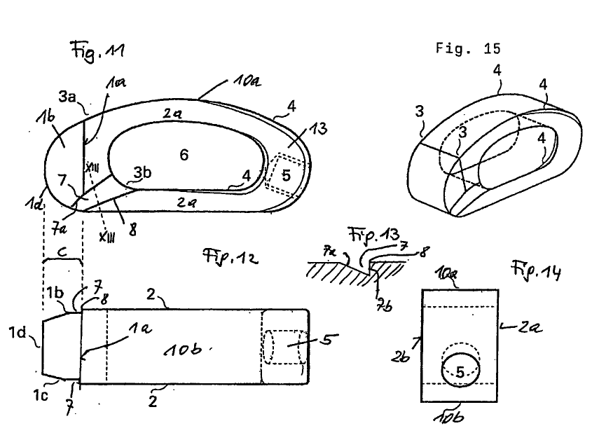

The section according to FIG. 9 shows primarily the front edges 3a, 3b, which become sharp at the wedge end 12 of the implant part D. They are formed by the surfaces 2a, 2b and the front surfaces of the front wall 2a and rear wall 2b of the implant part D. The front 2a and rear wall 2b enclose the central recess 6 passing through the implant from top to bottom. FIG. 10 shows in section the front edges 4, which become increasingly round towards the implant end 13 opposite the distraction wedge C. Incidentally, the section is to be described the same as the section shown in FIG. 4.

FIGS. 11 to 14 show an embodiment for an intervertebral distraction implant with guide grooves offset towards the back. The implant I comprises a pressure-receiving part D and distraction wedge C. In the projection, it has a convex front wall 10a, a straight rear wall 10b, and is rounded at the distraction wedge C and the opposite end 13. The implant walls enclose a central recess 6. The front edges of the implant part D become increasingly sharp (3a, 3b) towards the distraction wedge C and are increasingly rounded towards the opposite end 13. At this end 13 of the implant part D there is a bore 5 for attaching an insertion instrument. In the top surface 1b and bottom surface 1c of the distraction wedge C, as well as in the adjacent surfaces 2a, 2b of the implant part D, there are grooves 7 running in parallel or concentric to the front edges 3a. In this embodiment, each of the two grooves are offset towards the back, such that the front surface of the rear wall 10b of the implant part D transitions with a kink or a bend, but continuously, into the rear wall 7b of the groove and also the sharp groove edge 8 forms such a continuation of the sharp front edge 2b of the rear wall of the implant part D 10b. The rear wall of the groove shown here has a straight profile in the projection as another possible configuration. Its direction corresponds approximately to a horizontal tangent, which is positioned in the projection at the transition of the implant part D into the distraction wedge C to the curvature of the front wall 10a in this position.

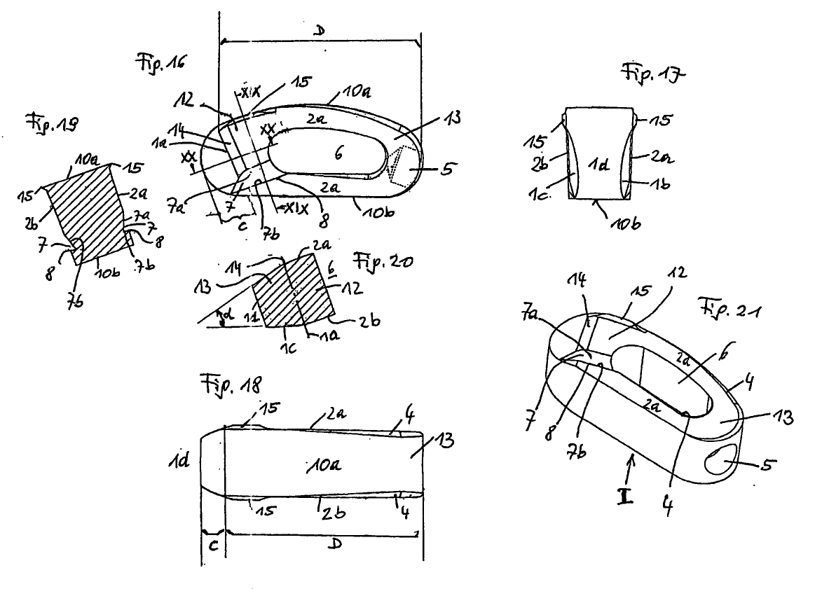

The projection according to FIG. 12 shows this intervertebral distraction implant from behind. The top 2a and bottom 2b implant surface run parallel to each other and are flat in this case. The wedge surfaces 1b, 1c define the angle α. The front ends of the grooves 7 are found on the wedge surfaces 1b, 1c. In the section shown in FIG. 13 through the portion of the guide groove 7 lying in the implant part D, each of the two grooves has the shape of an inverted lean-to roof with a vertical rear wall 7b, which forms, with the surface 2a and 2b of the implant part D, a sharp edge 8 pointing forwards and which extends forwards from the base of the groove at an angle relative to the surfaces 2a, 2b, or 1b, 1c. The projection according to FIG. 14 shows the end 13 of this intervertebral distraction implant opposite the distraction wedge C. Both the top 2a and bottom surface and also the front 10a and rear wall 10b of the implant part D are flat in this case and define a right angle with each other. Here, a device for attaching an insertion instrument is formed on this implant part 13. FIG. 15 shows the intervertebral distraction implant in a three-dimensional perspective view.

FIGS. 16 to 21 show an embodiment for an intervertebral distraction implant with distraction wedge C positioned at an angle and guide ridges formed on the sharp front edges. The implant I comprises a pressure-receiving part D and the distraction wedge C. In this case, the distraction wedge C is formed at an angle to the longitudinal axis (not shown) of the implant running parallel to the rear surface of the implant rear wall 2b, such that the base of the distraction wedge C encloses, with the longitudinal axis of the implant, an angle of approximately 110 degrees, which is open towards the front. In the projection, the implant has a convex front wall 10a, a straight rear wall 10b, and is rounded at the distraction wedge C and the opposite end 13. The device for attaching an insertion instrument is fixed at this end. The implant walls enclose a central recess 6. In this case, a sharp-edge guide ridge 15 extending continuously from the front surface 10a and projecting from the top surface 2a and bottom surface 2b of the implant is formed on the part connected to the distraction wedge C. These ridges 15 replace the sharp front edges 3a in the other configurations. In this configuration, only the wedge-side portions of the front edges of the rear wall have a sharp edge 3b. The front edges 4 of the walls of the implant part D also become round in this case towards the implant end 13 opposite the wedge part C. With regard to the localization and shape of the guide grooves 7, refer to FIGS. 11 to 15 and their description.

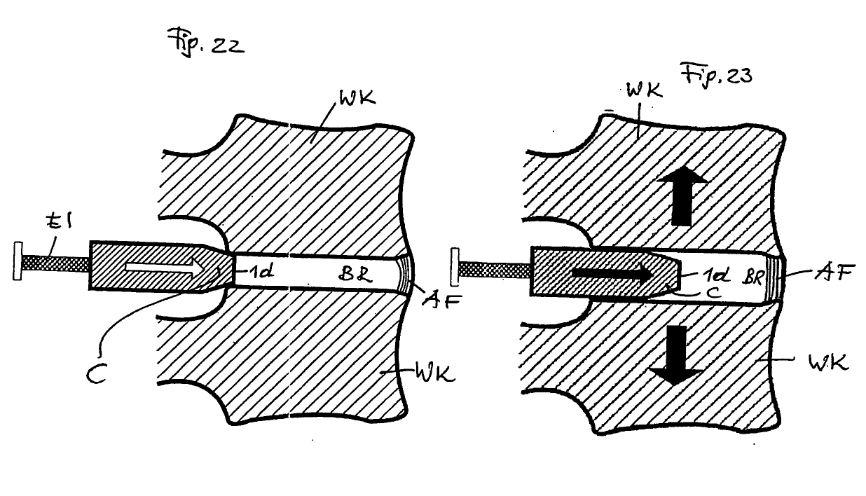

The profile projection of the distraction wedge C according to FIG. 17 illustrates the sharp-edged guide ridges 15 extending from the front wall 10a of the implant part D. FIG. 18 shows a projection of the implant from the front. The representation shows the guide ridges 15 and their relationship to the front wall 10a, as well as the surfaces 2a, 2b, and the edges 4 of the implant. The guide ridges 15 begin at the transition of the distraction wedge C to the implant part D and from there extend no farther than 10 mm in the direction towards the implant end 13 opposite the wedge end.

The cross section according to FIG. 19 shows the wedge-side portion 12 of the implant part D, how the sharp guide ridges 15 project over the implant part D and extend continuously from the front wall 10a of this implant part. The front surfaces of the guide ridges 15 having a lean-to roof-shaped cross section form a continuation of the implant front wall 10a. These front surfaces form a sharp edge with the surfaces falling towards the back at an angle relative to the surfaces 2a, 2b of the implant. Each of the two guide ridges should project over the implant part D by no more than 0.8 mm. Also shown are the guide grooves 7 with their vertical rear walls 7b, the front surfaces 7a rising at an angle towards the front, and the sharp edges 8 formed by the rear walls 7b with the corresponding surface of the implant part D 2a, 2b. According to FIG. 20, the section guided vertical relative to the base la of the distraction wedge C and the adjacent part 12 of the implant part D illustrates the distraction wedge C, which has already been described several times. It is composed of a base 1a made from the two wedge surfaces 1b, 1c and the wedge end 1d. The wedge base 1a transitions uninterrupted to the adjacent part 12 of the implant part D, which, on its side, forms the wedge-side wall of the central recess 6. The two wedge surfaces 1b, 1c are inclined at an angle α relative to each other and transition at the wedge base with an elbow into the adjacent surfaces 2a, 2b of the wedge-side portion 12 of the implant part D. The relevant transition can have a small radius 14 (cf. FIG. 16). The three-dimensional illustration according to FIG. 21 shows the shape of the intervertebral implant I according to the invention.

FIGS. 22 and 23, schematic representations of the distraction mechanism show how the vertical distance of the vertebrae can be expanded (distracted) with the help of an intervertebral distraction implant according to the invention. The implant is positioned according to FIG. 22. The intervertebral-disc space BR is opened from the dorsal or dorsolateral side through the annulus fibrosus AF in the shown case, so that the provided implant can be inserted through the opening. The intervertebral-disc space is completely cleaned out up to the annulus fibrosus. An implant is selected, whose front wedge end 1d is ca. 1 mm lower than the vertical distance of the vertebrae running from top to bottom and defined by the facing end plates of the vertebrae and thus can be inserted straight into the intervertebral-disc space BR. The implant is inserted into the intervertebral-disc space according to FIG. 23. With the help of the insertion instrument EI attached to the implant, the implant is inserted into the intervertebral-disc space. As soon as the distraction wedge C of the implant has been inserted completely into the intervertebral-disc space as described here, the vertical distance of the vertebrae is enlarged, i.e., distracted in the direction of the two arrows, to the implant height in the direction of the two arrows. Because discs connecting the vertebra and also the annulus fibrosus are tensioned during the distraction, the resistance to be overcome during the insertion, especially in the end phase of the distraction, can be large enough that the implant can no longer be inserted manually. It must be hammered into the intervertebral-disc space through dosed hammer strikes on the handle of the insertion instrument (EI). In the sense of the invention, it is desired that the implant be clamped tightly between the vertebrae by the tension of the mentioned discs and the annulus fibrosus.

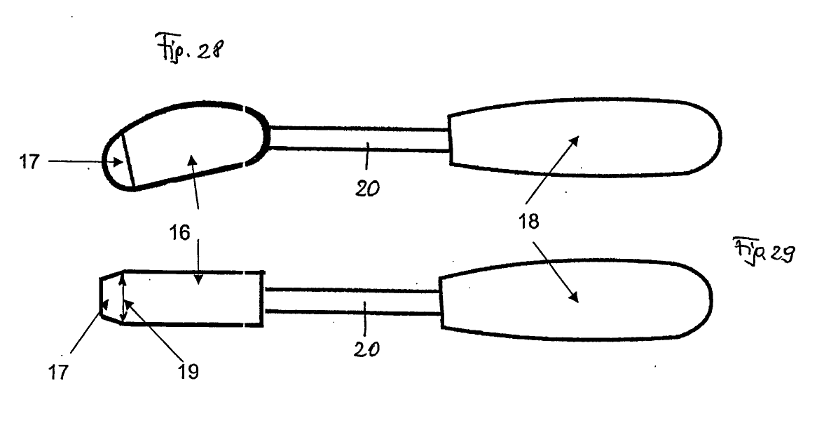

FIGS. 24 to 27 show the implantation of an intervertebral distraction implant. In each of the figures, the top vertebra is removed. The top to the bottom vertebra W can be seen. The intervertebral foramina are located between the joint projections GF and the portion of the annulus fibrosus AF at the back on the side. The intervertebral disc is opened from the outside of the intervertebral foramina only so far that the implant I can be inserted at the back on the side through the opening into the intervertebral-disc space and the intervertebral-disc space BR can be completely cleaned out up to the annulus fibrosus AF through this same opening. In phase 1 according to FIG. 24, the implant I attached to the insertion instrument EI is already inserted with the distraction wedge C in the direction of the implant longitudinal axis IA into the intervertebral-disc space BR so far that the distraction is completed. In this phase, the implant I is already jammed in the intervertebral-disc space. From this phase on, the implant must be guided by the guide element formed on the implant towards the end position shown in FIG. 27. As follows from the figure, the insertion instrument EI is attached to the implant I, such that its shaft encloses, with the longitudinal axis of the implant, an angle of 25 degrees to 30 degrees. Therefore, it is possible to pivot the implant I with the help of the insertion instrument EI, whose shaft nearly contacts the joint projection GF at the beginning, also approximately in the direction (no arrow) of the end position. ER designates the implantation direction given by the insertion instrument EI. In phase II shown in FIG. 25, the implant is inserted further into the intervertebral-disc space with the help of the insertion instrument EI. The implant I rotates further in the direction of the end position thanks to the guide element in the intervertebral-disc space BR. Here, the shaft of the insertion instrument EI is pivoted towards the side so far that it reached the lateral limit of the access to the vertebral column (not shown) and therefore must be retracted.

In phase III (FIG. 26), the implant is hammered farther into the intervertebral-disc space BR, after the retraction of the insertion instrument EI, with the help of a striking ram ES positioned on its end opposite the distraction wedge C. Due to the lateral limit of the access to the vertebral column, the insertion or striking direction ER also cannot pivot farther to the side with the striking ram. However, the end of the striking ram ES contacting the implant I is shaped such that it does not obstruct the further rotation of the implant caused by the guide element formed on the implant. Phase IV is shown in FIG. 27. The implant I, hammered with the striking ram ES, is rotated by further impacts into its end position thanks to the guide element. The longitudinal axis of the implant IA now lies in the frontal plane. The striking ram is retracted and the spondylodesis is completed through the introduction of autological material or bone replacement material into the space, which is located between the implant I and the rear portion of the annulus fibrosus AF in addition to the autological spongiosa filled into the central recess 6 of the implant or the bone replacement material filled into the recess 6.

FIGS. 28 and 29 show a distraction instrument. Such instruments are used for the aforementioned distraction possibly required for inserting the intervertebral distraction implant according to the invention into the intervertebral space. The instrument comprises a compact body 16, a shaft 20, and a handle 18. The shaft 20 can be connected rigidly to the body 16 or can be removable from this body. The body 16 has a shape that is similar to the intervertebral distraction implants. Its front part 17 is shaped like the distraction wedge C of an intervertebral distraction implant. The surfaces of the body 16 are smooth. Distraction instruments with different body heights 19 are provided. The intervals of the body heights 19 should preferably equal 3 mm and should be dimensioned such that the body 18 of a distraction instrument is higher by 1 mm than the front end 1d of the distraction wedge C of a matching intervertebral distraction implant. Thus it is ensured that the distraction wedge C of the corresponding intervertebral implant can be inserted into the intervertebral space shown in FIG. 22. If it is provided that the shaft 20 can be removed from the body 16, then it is sufficient to provide only a series of bodies 16 with different heights instead of a series of distraction instruments. The axis of the shaft 20 defines, with the longitudinal axis of the body 16 running parallel to the straight rear wall of the body 16, an angle of 25 degrees to 30 degrees.

The following is a series of special features according to the invention, which are again clearly listed:

The intervertebral distraction implants of the invention are used for stabilizing interbody spondylodesis. They transfer forces acting on the corresponding upper vertebra to the lower vertebra and ensure that, between the two vertebrae and the implant, there are no movements that could disrupt the formation of a solid osseous connection between the two vertebrae.

Such implants can be used as cages for bone transplant material or bone replacement material if they have a recess 6 that is continuous from the cranial side towards the coccygeal side. However, they can also be compact, i.e., they can have no recess. In this case, material promoting the osseous growth of the spondylodesis is placed around the implant.

Intervertebral distraction implants according to the invention, which are adapted to the anatomical conditions and the application technique, can be inserted dorsally, dorsolaterally, or laterally into the intervertebral-disc spaces of the thoracic and lumbar vertebral column.

Through the dimensioning and shape of the implants according to the invention, the goal is achieved of being able to insert the largest possible implants with especially large contact surfaces to the vertebrae into the intervertebral-disc space, so that the surface pressure in the region of the contact surfaces of the vertebrae is reduced. Thus there is less risk that the vertebrae will fracture in those areas. In addition, the central recess receiving the bone or bone replacement material should also be as large as possible.

Furthermore, by the special shape of the intervertebral distraction implants according to the invention, the goal is achieved of being able to produce the enlargement of the vertical distance between the two vertebrae (distraction), which is advantageous for the spondylodesis in two respects, just with the implant itself. First, distraction leads to the tensioning of the discs and the intervertebral-disc ring connecting the two vertebrae. This creates the tight lodging of the implant, which is crucial for the stability of the spondylodesis, between the two vertebrae. Second, the distraction causes an expansion of the spinal canal and the intervertebral foramina and thus a decompression of the nerve tracts through the spinal canal and the foramina.

Consequently, the intervertebral distraction implants according to the invention comprise two parts in terms of function, a wedge-shaped insertion part (distraction wedge C) and a pressure-transmitting implant part D.

With the distraction wedge C, the vertical distance of the vertebrae is enlarged by an amount corresponding to the difference between the wedge end and the wedge base when the implant is inserted into the intervertebral wedge space. The angle defined by the wedge surfaces should not be significantly larger than 30 degrees, so that the edges of the vertebrae will not fracture when the wedge is inserted.

Special distractors (FIGS. 28 and 29) can be used for preparing the optimal distraction.

The cylindrical or prismatic pressure-transmitting implant part D can have an opening 6, which is continuous in the vertical direction and which is used for receiving bone or bone replacement material. However, it can also be compact. Its lateral walls can be closed or have through openings from the outside towards the inside. The surfaces facing the vertebrae can be flat or curved, as well as parallel or inclined relative to each other. A device for attaching an insertion instrument can be formed on the part of the implant facing away from the insertion part.

The intervertebral distraction implants according to the invention can be equipped with special guide elements for simplifying their ability to rotate in the intervertebral-disc space. On the distraction wedge C, these guide elements can comprise a specially shaped groove 7 or from such a groove with a ridge 9 positioned on the groove. On the actual implant body, the implant part D, the guide elements comprise the front edges 3a, 3b, which become sharper towards the wedge end, the optional sharp-edged ridges 15 positioned on the front edge 3a, the grooves 7 extending from the distraction wedge into the adjacent part of the implant body, and optional ridges 9 positioned on these grooves.

For the implantation of the intervertebral implant, e.g., a rod-shaped insertion instrument EI with a handle as well as a ram ES can be provided, whose front end has the same rounding as the rear end 13 of the implant.

The implants can be made from metal, polymer, or a composite material. The surfaces of the implants can be smooth, structured, or coated. Structures, such as, e.g., small cones, prisms, or ridges, projecting from the surfaces 2a, 2b of an implant according to the invention can be arranged such that they form rows running in parallel or concentric to the front edges of the implant, so that they can also act as guide elements.

Elements or materials casting radiological shadows are provided into implants made from polymer or composite material, in order to make them visible to X-ray imaging.

List of numbers and abbreviations used in the drawings and in the description
1a Base of distraction wedge
1b Top surface of distraction wedge
1c Bottom surface of distraction wedge
1d Front end of distraction wedge
2a Top surface of implant part D
2b Bottom surface of implant part D
3a, 3b Sharp front edges of implant part D
4 Rounded front edge surface of implant part D
5 Device for attaching an insertion instrument
6 Central recess of surface of implant part D
7 Guide groove in distraction wedge C and implant part D
7a Vertical rear wall of guide groove 7
7b Groove surface extending from the base of groove 7 at an angle relative to surfaces 1b and 1c
8 Sharp rear edge of guide groove 7
9 Sharp-edged ridge positioned on 7a
10a Front wall of implant
10b Rear wall of implant
12 Wedge-side end of implant part D
13 End of implant part D opposite the wedge-side end
14 Rounded transition of wedge surfaces 1b and 1c into the implant surfaces 2a and 2b
15 Sharp-edged ridge positioned close to the distraction wedge C on the front edges 3a
16 Body of a distractor
17 Wedge of a distractor body
18 Handle of a distractor body
19 Height of a distractor body
AF Annulus fibrosus, intervertebral-disc ring
BR Intervertebral-disc space
C Distraction wedge
D Pressure-transmitting implant part
EI Insertion instrument
ER Insertion and striking direction
ES Striking ram
GF Joint projection
I Implant body
IA Implant longitudinal axis
SR Pivot direction of the insertion instrument
W Vertebra
WK Vertebra body

1. Implant for stabilizing operations on the thoracic and lumbar vertebral column for restoring the load-bearing capacity of the vertebral column, wherein a part of the implant has a wedge-shaped bevel, which enables vertebrae of the vertebral column to be pressed apart for enlarging the intervertebral distance.

2. Implant according to claim 1, wherein the implant is comprises a wedge-shaped insertion part and a pressure-transmitting implant part.

3. Implant according to claim 1, wherein an angle defined by the wedge surfaces equals at least approximately 30°, preferably 25° to 30°.

4. Implant according to claim 1, wherein base, cover, and side surfaces of the implant are flat or slightly curved and in parallel or inclined relative to each other.

5. Implant according to claim 1, wherein surfaces of the implant are smooth, structured, or coated.

6. Implant according to claim 5, wherein surfaces of the implant are equipped with projecting structures, which form rows running in parallel or concentric to front edges of the implant.

7. Implant according to claim 1, wherein elements or substances casting radiological shadows are provided in the implant when material forming the implant itself does not cast shadows radiologically.

8. Implant according to claim 1, further comprising a device for attaching an implantation instrument.

9. Implant according to claim 1, further comprising a first end part which has a convex curve and a second end part which is flat, convex, or concave, and the two end parts are rounded cylindrically, wherein one of the end parts, with which the implant can be inserted into the intervertebral-disc space, is longer than the other end part and wherein a part of the top and/or bottom surface of the longer part features comprises a wedge-shaped bevel.

10. Implant according to claim 9, wherein surfaces that run approximately parallel to outer walls of the implant define a central recess.

11. Implant according to claim 9, further comprising with a central recess, and convex side, top and bottom front edges of front and rear walls of the implant begin with sharp edges on the side of the wedge-shaped end and are rounded increasingly towards the other end.

12. Implant according to claim 2, wherein guide elements in the form of grooves or ridges are provided in at least one of the wedge-shaped end part or the implant part.

13. Implant according to claim 12, further comprising sharp-edged grooves, which are parallel or concentric to sharp front edges, and are arranged into the top and bottom surface of the pressure-transmitting implant part adjacent to the recess on the wedge side, wherein the grooves or the ridges positioned on the sharp-edged grooves also have a straight-line profile.

14. Implant according to claim 12, wherein the grooves have the shape of an inverted lean-to roof-shaped, two-surface groove, whose rear surface is vertical to a surface of the implant and forms a sharp edge with this surface of the implant and whose front surface extends at an angle relative to the surface of the implant.

15. Implant according to claim 14, wherein the sharp edges of the grooves run in parallel or concentric to a curvature of the corresponding portions of front edges of a front wall of the implant.

16. Implant according to claim 12, wherein edges of the grooves are configured as straight lines and extend at an angle through a surface of the implant, such that their direction lies parallel to a tangent, which is positioned at the transition of the cylindrical implant part into the wedge-shaped implant part to a curvature of the front surface of the implant.

17. Implant according to claim 6, wherein the projecting structures comprise at least one of small cones, prisms, or ridges.

18. Implant according to claim 9, wherein convex-side top and bottom front edges of front wall of the implant begin with sharp edges on the side of the wedge-shaped end and are rounded increasingly towards the other.
